(19) United States
(12) Patent Application Publication
(10) Pub. No.: US 2009/0299650 A1
(43) Pub. Date: Dec. 3, 2009

(54) SYSTEMS AND METHODS FOR FILTERING TARGET PROBE SETS

(76) Inventors: Charles F. Nelson, Loveland, CO (US); Jing Gao, Loveland, CO (US); Amitabh Shukla, Loveland, CO (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 12/130,924

(22) Filed: May 30, 2008

(51) Int. Cl. G01N 33/50; C12Q 1/68; G06F 17/30

(52) U.S. Cl. 702/20; 435/6; 707/102; 707/E17.046

(57) ABSTRACT

Systems and methods for using the same to filter target probes sets are provided. In certain embodiments, the system and methods are implemented on a web-based platform. Also provided are computer program products for executing the subject methods.

### BACKGROUND

Biomolecular probes, such as nucleic acids and polypeptides, have become an increasingly important tool in the biotechnology industry and related fields. For a biomolecular probe to be of use in a particular binding assay, it needs to have associated with it specific information, e.g., its target binding specificity. This information is generally referred to as probe annotation.

One area in which annotated biomolecular probes are of particular use is in the generation and use of biopolymeric arrays. Biopolymeric arrays include regions of usually different sequence annotated probes arranged in a predetermined configuration on a substrate. These regions (sometimes referenced as “features”) are positioned at respective locations (“addresses”) on the substrate. The arrays, when exposed to a sample, will exhibit an observed binding pattern which can be detected upon interrogating the array. By correlating the observed binding pattern with the known locations of the annotated biopolymeric probes on the array, one can determine the presence and/or concentration of one or more probe-binding components of the sample.

### SUMMARY OF THE INVENTION

Systems and methods for using the same to filter target probe sets are provided. In providing such systems and methods

Aspects of the present invention include a system for selecting a probe group including (A) a communication module comprising an input manager for receiving input from a user and an output manager for communicating output to a user; (B) a processing module including: (1) a probe design manager configured to design a probe set, wherein the probe set includes probes specific for one or more target nucleic acid sequences; and (2) a probe selection manager configured to: (a) select a probe group from the probe set using a probe filter, where the probe filter includes a set of rules for selecting the probe group; and (b) communicate the selected probe set to the user; where the system is implemented on a web-based platform.

In certain embodiments, the set of rules defines one or more of the following: probe length parameter, probe distribution parameter, probes per target, probe melting temperature (Tm), base composition parameter, sequence quality score, X base %, thermodynamic properties, intramolecular hybridization characteristics, intermolecular hybridization characteristics, cross-hybridization potential, validation status, biological characteristic, and hybridization assay parameter.

In certain embodiments, the system further includes a probe filter database comprising a plurality of probe filters, wherein the probe filter is selected from the probe filter database.

In certain embodiments, the plurality of probe filters in the probe filter database are provided to the system by any combination of: the user, an administrator of the system, and any other user of the system.

In certain embodiments, the system further includes a security manager configured to allow a probe filter to be modified or deleted only by the user or administrator who provided the probe filter to the system.

In certain embodiments, the system further includes a graphical user interface (GUI), wherein the probe filter is selected by the user by engaging one or more graphical icons present on the GUI.

In certain embodiments, the one or more graphical icons include: drop-down menus, buttons, text strings, fill-in boxes, arrow buttons, and hyperlinks.

In certain embodiments, the probe filter employed is selected by the user.

In certain embodiments, the probe filter employed is a default filter.

In certain embodiments, the system is further configured to select a second probe group from the selected probe group using a second probe filter when prompted by the user.

In certain embodiments, the system further includes a user domain manager configured to save the selected probe group in a user domain specific for the user when prompted by the user.

In certain embodiments, the probe set is saved in the user domain when prompted by the user.

In certain embodiments, the processing module further includes an array layout developer configured to develop an array layout comprising the selected probe group.

Aspects of the invention include methods of receiving a selected probe set including: (a) providing one or more target nucleic acids to the system of claim 1; and (b) receiving a selected probe group for the one or more targets.

Aspects of the invention include methods of providing a probe group to a user, the method comprising:(a) designing a probe set based on input received from a user, wherein the probe set includes probes specific for one or more target nucleic acid sequences; (b) selecting a probe group from the probe set using a probe filter, wherein the probe filter is: (i) selected by the user; and (ii) includes a set of rules for selecting the probe group; (c) providing the probe group to the user, where the method is carried out on a web-based platform.

In certain embodiments, the set of rules defines one or more of the following: probe length parameter, probe distribution parameter, probes per target, probe melting temperature (Tm), base composition parameter, sequence quality score, X base %, thermodynamic properties, intramolecular hybridization characteristics, intermolecular hybridization characteristics, cross-hybridization potential, validation status, biological characteristic, and hybridization assay parameter.

In certain embodiments, the probe filter is selected from a probe filter database comprising a plurality of probe filters.

In certain embodiments, each of the plurality of probe filters in the probe filter database is provided by any combination of: the user, an administrator of the system, and any other user of the system.

In certain embodiments, the web-based platform includes a graphical user interface (GUI) that displays graphical icons to the user, wherein the user engages one or more of the graphical icons to select the probe filter.

In certain embodiments, the one or more graphical icons include: drop-down menus, buttons, text strings, fill-in boxes, arrow buttons, and hyperlinks.

In certain embodiments, the method further includes selecting a second probe group from the selected probe group using a second probe filter when prompted by the user.

In certain embodiments, the selected probe group is saved in a user domain specific for the user when prompted by the user.

In certain embodiments, the probe set is saved in the user domain when prompted by the user.

In certain embodiments, the method further includes developing an array layout comprising the selected probe group when prompted by the user.

In certain embodiments, the method further includes fabricating an array based on the array layout and sending the fabricated array to the user.

Aspects of the invention include computer program products comprising a computer readable storage medium having a computer program stored thereon, where the computer program, when loaded onto a computer, operates the computer to carry out the probe filter methods of the present invention on a web-based platform.

### BRIEF DESCRIPTIONS OF THE DRAWINGS

FIG. 1 illustrates a substrate carrying multiple arrays, such as may be fabricated by methods of the present invention.

FIG. 2 is an enlarged view of a portion of FIG. 1 showing multiple ideal spots or features.

FIG. 3 is an enlarged illustration of a portion of the substrate in FIG. 2.

FIG. 4 schematically illustrates an exemplary system of the present invention.

FIG. 5 provides an exemplary embodiment of a user interface schematic presented on a graphical user interface (GUI) that can be used in conjunction with the target probe filter systems and methods of the present invention.

### DEFINITIONS

Unless defined otherwise, all technical and scientific terms used herein have the same meaning as commonly understood by one of ordinary skill in the art to which this invention belongs. Still, certain elements are defined below for the sake of clarity and ease of reference.

By “array layout” is meant a collection of information, e.g., in the form of a file, which represents the location of probes that have been assigned to specific features of one or more array formats, e.g., a single array format or two or more array formats of an array set.

The phrase “array format” refers to a format that defines an array by feature number, feature size, Cartesian coordinates of each feature, and distance that exists between features within a given single array.

The phrase “array content information” is used to refer to any type of information/data that describes an array. Representative types of array content information include, but are not limited to: “probe-level information” and “array-level information”. By “probe-level information” is meant any information relating to the biochemical properties or descriptive characteristics of a probe. Examples include, but are not limited to: probe sequence, melting temperature (Tₘ), target gene or genes (e.g., gene name, accession number, etc.), location identifier information, information regarding cell(s) or tissue(s) in which a probe sequence is expressed and/or levels of expression, information concerning physiological responses of a cell or tissue in which the sequence is expressed (e.g., whether the cell or tissue is from a patient with a disease), chromosomal location information, copy number information, information relating to similar sequences (e.g., homologous, paralogous or orthologous sequences), frequency of the sequence in a population, information relating to polymorphic variants of the probe sequence (e.g., such as SNPs), information relating to splice variants (e.g., tissues, individuals in which such variants are expressed), demographic information relating to individual(s) in which the sequence is found, and/or other annotation information. By “array-level information” is meant information relating to the physical properties or intended use of an array. Examples include, but are not limited to: types of genes to be studied using the array, such as genes from a specific species (e.g., mouse, human), genes associated with specific tissues (e.g., liver, brain, cardiac), genes associated with specific physiological functions, (e.g., apoptosis, stress response), genes associated with disease states (e.g., cancer, cardiovascular disease), array format information, e.g., feature number, feature size, cartesian coordinates of each feature, and distance that exists between features within a given array, etc.

A “data element” represents a property of a probe sequence, which can include the base composition of the probe sequence. Data elements can also include representations of other properties of probe sequences, such as expression levels in one or more tissues, interactions between a sequence (and/or its encoded products), and other molecules, a representation of copy number, a representation of the relationship between its activity (or lack thereof) in a cellular pathway (e.g., a signaling pathway) and a physiological response, sequence similarity to other probe sequences, a representation of its function, a representation of its modified, processed, and/or variant forms, a representation of splice variants, the locations of introns and exons, functional domains etc. A data element can be represented for example, by an alphanumeric string (e.g., representing bases), by a number, by “plus” and “minus” symbols or other symbols, by a color hue, by a word, or by another form (descriptive or nondescriptive) suitable for computation, analysis and/or processing for example, by a computer or other machine or system capable of data integration and analysis.

As used herein, the term “data structure” is intended to mean an organization of information, such as a physical or logical relationship among data elements, designed to support specific data manipulation functions, such as an algorithm. The term can include, for example, a list or other collection type of data elements that can be added, subtracted, combined or otherwise manipulated. Exemplary types of data structures include a list, linked-list, doubly linked-list, indexed list, table, matrix, queue, stack, heap, dictionary, flat file databases, relational databases, local databases, distributed databases, thin client databases and tree. The term also can include organizational structures of information that relate or correlate, for example, data elements from a plurality of data structures or other forms of data management structures. A specific example of information organized by a data structure of the invention is the association of a plurality of data elements relating to a gene, e.g., its sequence, expression level in one or more tissues, copy number, activity states (e.g., active or non-active in one or more tissues), its modified, processed and/or and/or variant forms, splice variants encoded by the gene, the locations of introns and exons, functional domains, interactions with other molecules, function, sequence similarity to other probe sequences, etc. A data structure can be a recorded form of information (such as a list) or can contain additional information (e.g., annotations) regarding the information contained therein. A data structure can include pointers or links to resources external to the data structure (e.g., such as external databases). In one aspect, a data structure is embodied in a tangible form, e.g. is stored or represented in a tangible medium (such as a computer readable medium).

The term “object” refers to a unique concrete instance of an abstract data type, a class (that is, a conceptual structure including both data and the methods to access it) whose identity is separate from that of other objects, although it can “communicate” with them via messages. In some occasions, some objects can be conceived of as a subprogram which can communicate with others by receiving or giving instructions based on its, or the others' data or methods. Data can consist of numbers, literal strings, variables, references, etc. In addition to data, an object can include methods for manipulating data. In certain instances, an object may be viewed as a region of storage. In the present invention, an object typically includes a plurality of data elements and methods for manipulating such data elements.

A “relation” or “relationship” is an interaction between multiple data elements and/or data structures and/or objects. A list of properties may be attached to a relation. Such properties may include name, type, location, etc. A relation may be expressed as a link in a network diagram. Each data element may play a specific “role” in a relation.

As used herein, an “annotation” is a comment, explanation, note, link, or metadata about a data element, data structure or object, or a collection thereof. Annotations may include pointers to external objects or external data. An annotation may optionally include information about an author who created or modified the annotation, as well as information about when that creation or modification occurred. In one embodiment, a memory comprising a plurality of data structures organized by annotation category provides a database through which information from multiple databases, public or private, may be accessed, assembled, and processed. Annotation tools include, but are not limited to, software such as BioFerret (available from Agilent Technologies, Inc., Palo Alto, Calif.), which is described in detail in U.S. patent application Ser. No. 10/033,823 filed Dec. 19, 2001 and titled “Domain-Specific Knowledge-Based Metasearch System and Methods of Using”, which is incorporated herein by reference in its entirety. Such tools may be used to generate a list of associations between genes from scientific literature and patent publications.

As used herein an “annotation category” is a human readable string to annotate the logical type that an object comprising its plurality of data elements represents. Data structures that contain the same types and instances of data elements may be assigned identical annotations, while data structures that contain different types and instances of data elements may be assigned different annotations.

As used herein, a “probe sequence identifier” or an “identifier corresponding to a probe sequence” refers to a string of one or more characters (e.g., alphanumeric characters), symbols, images or other graphical representation(s) associated with a probe sequence comprising a probe sequence such that the identifier provides a “shorthand” designation for the sequence. In one aspect, an identifier comprises an accession number or a clone number. An identifier may comprise descriptive information. For example, an identifier may include a reference citation or a portion thereof.

The term “filter” or “probe filter” (and equivalents) refers to one or more rules (also called “filter criteria”) defining a limit, range, criterion or hierarchical ranking for a probe parameter. When a filter is applied to a probe set (either designed by the system of the present invention or provided by a user of the system), probes that satisfy the one or more rules are returned to the user (e.g., as a file, displayed in a GUI, saved in a user domain, etc.). In certain embodiments, the probes returned are called a probe group. In certain embodiments, a previously filtered probe group may itself be subjected to further filtering using a different filter, generating a second probe group. In certain embodiments, a filter can be provided to the subject systems by one or more users or administrators of the system. A plurality of filters can be present in a filter database of the subject systems from which a user of the system can select to apply to a probe set. In certain embodiments, a user specifies filter criteria (or rules) immediately prior to application to a probe set without saving the filter in the filter database (e.g., using any convenient input mode on a GUI of the system).

The term “best-fit” or “best” refers to a resource allocation scheme that determines the best result in response to input data. The definition of ‘best’ may vary depending on a given set of parameters examined, with exemplary parameters including (but not limited to) sequence identity limits, signal intensity limits, cross-hybridization limits, melting temperature (Tₘ), base composition limits, probe length limits, distribution of bases along the length of the probe, distribution of nucleation points along the length of the probe (e.g., regions of the probe likely to participate in hybridization), secondary structure parameters, etc. In one aspect, the system considers predefined thresholds. In another aspect, the system rank-orders fit. In a further aspect, the user defines his or her own thresholds, which may or may not include system-defined thresholds.

The terms “system” and “computer-based system” refer to the hardware means, software means, and data storage means used to analyze the information of the present invention. The minimum hardware of the computer-based systems of the present invention comprises a central processing unit (CPU), input means, output means, and data storage means. As such, any convenient computer-based system may be employed in the present invention. The data storage means may comprise any manufacture comprising a recording of the present information as described above, or a memory access means that can access such a manufacture.

A “processor” references any hardware and/or software combination which will perform the functions required of it. For example, any processor herein may be a programmable digital microprocessor such as available in the form of an electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

“Computer readable medium” as used herein refers to any storage or transmission medium that participates in providing instructions and/or data to a computer for execution and/or processing. Examples of storage media include floppy disks, magnetic tape, USB, CD-ROM, a hard disk drive, a ROM or integrated circuit, a magneto-optical disk, or a computer readable card such as a PCMCIA card and the like, whether or not such devices are internal or external to the computer. A file containing information may be “stored” on computer readable medium, where “storing” means recording information such that it is accessible and retrievable at a later date by a computer. A file may be stored in permanent memory.

With respect to computer readable media, “permanent memory” refers to memory that is permanently stored on a data storage medium. Permanent memory is not erased by termination of the electrical supply to a computer or processor. Computer hard-drive ROM (i.e. ROM not used as virtual memory), CD-ROM, floppy disk and DVD are all examples of permanent memory. Random Access Memory (RAM) is an example of non-permanent memory. A file in permanent memory may be editable and re-writable.

To “record” data, programming or other information on a computer readable medium refers to a process for storing information, using any convenient method. Any convenient data storage structure may be chosen, based on the means used to access the stored information. A variety of data processor programs and formats can be used for storage, e.g. word processing text file, database format, etc.

A “memory” or “memory unit” refers to any device which can store information for subsequent retrieval by a processor, and may include magnetic or optical devices (such as a hard disk, floppy disk, CD, or DVD), or solid state memory devices (such as volatile or non-volatile RAM). A memory or memory unit may have more than one physical memory device of the same or different types (for example, a memory may have multiple memory devices such as multiple hard drives or multiple solid state memory devices or some combination of hard drives and solid state memory devices).

In certain embodiments, a system includes hardware components which take the form of one or more platforms, e.g., in the form of servers, such that any functional elements of the system, i.e., those elements of the system that carry out specific tasks (such as managing input and output of information, processing information, etc.) of the system may be carried out by the execution of software applications on and across the one or more computer platforms represented of the system. The one or more platforms present in the subject systems may be any convenient type of computer platform, e.g., such as a server, main-frame computer, a work station, etc. Where more than one platform is present, the platforms may be connected via any convenient type of connection, e.g., cabling or other communication system including wireless systems, either networked or otherwise. Where more than one platform is present, the platforms may be co-located or they may be physically separated. Various operating systems may be employed on any of the computer platforms, where representative operating systems include Windows, MacOS, Sun Solaris, Linux, OS/400, Compaq Tru64 Unix, SGI IRIX, Siemens Reliant Unix, and others. The functional elements of system may also be implemented in accordance with a variety of software facilitators, platforms, or other convenient method.

Items of data are “linked” to one another in a memory when the same data input (for example, filename or directory name or search term) retrieves the linked items (in a same file or not) or an input of one or more of the linked items retrieves one or more of the others.

The term “monomer” as used herein refers to a chemical entity that can be covalently linked to one or more other such entities to form a polymer. Of particular interest to the present application are nucleotide “monomers” that have first and second sites (e.g., 5′ and 3′ sites) suitable for binding to other like monomers by means of standard chemical reactions (e.g., nucleophilic substitution), and a diverse element which distinguishes a particular monomer from a different monomer of the same type (e.g., a nucleotide base, etc.). In general, synthesis of nucleic acids of this type utilizes an initial substrate-bound monomer that is used as a building-block in a multi-step synthesis procedure to form a complete nucleic acid. A “biomonomer” references a single unit, which can be linked with the same or other biomonomers to form a biopolymer (e.g., a single amino acid or nucleotide with two linking groups, one or both of which may have removable protecting groups).

The terms “nucleoside” and “nucleotide” are intended to include those moieties which contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

As used herein, the term “amino acid” is intended to include not only the L, D- and nonchiral forms of naturally occurring amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine), but also modified amino acids, amino acid analogs, and other chemical compounds which can be incorporated in conventional oligopeptide synthesis, e.g., 4-nitrophenylalanine, isoglutamic acid, isoglutamine, ε-nicotinoyl-lysine, isonipecotic acid, tetrahydroisoquinoleic acid, α-aminoisobutyric acid, sarcosine, citrulline, cysteic acid, t-butylglycine, t-butylalanine, phenylglycine, cyclohexylalanine, β-alanine, 4-aminobutyric acid, and the like.

The term “oligomer” is used herein to indicate a chemical entity that contains a plurality of monomers. As used herein, the terms “oligomer” and “polymer” are used interchangeably, as it is generally, although not necessarily, smaller “polymers” that are prepared using the functionalized substrates of the invention, particularly in conjunction with combinatorial chemistry techniques. Examples of oligomers and polymers include polydeoxyribonucleotides (DNA), polyribonucleotides (RNA), other polynucleotides which are C-glycosides of a purine or pyrimidine base, polypeptides (proteins), polysaccharides (starches, or polysugars), and other chemical entities that contain repeating units of like chemical structure. In the practice of the instant invention, oligomers will generally comprise about 2-50 monomers, preferably about 2-20, more preferably about 3-10 monomers.

The term “polymer” means any compound that is made up of two or more monomeric units covalently bonded to each other, where the monomeric units may be the same or different, such that the polymer may be a homopolymer or a heteropolymer. Representative polymers include peptides, polysaccharides, nucleic acids and the like, where the polymers may be naturally occurring or synthetic.

A “biopolymer” is a polymer of one or more types of repeating units. Biopolymers are typically found in biological systems (although they may be made synthetically) and may include peptides or polynucleotides, as well as such compounds composed of or containing amino acid analogs or non-amino acid groups, or nucleotide analogs or non-nucleotide groups. This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. For example, a “biopolymer” may include DNA (including cDNA), RNA, oligonucleotides, and PNA and other polynucleotides as described in U.S. Pat. No. 5,948,902 and references cited therein (all of which are incorporated herein by reference), regardless of the source.

The term “biomolecular probe” or “probe” means any organic or biochemical molecule, group or species of interest having a particular sequence or structure. In certain embodiments, a biomolecular probe may be formed in an array on a substrate surface. Exemplary biomolecular probes include polypeptides, proteins, oligonucleotide and polynucleotides. In certain embodiments, a probe is designed to bind specifically to a target under appropriate assay conditions (e.g., stringent hybridization conditions), where such a probe may be employed to detect the presence or absence of its corresponding target in a sample. In certain embodiments a probe is designed not to bind to any target present in a sample (e.g., a negative control probe).

The term “probe set” means a set of probes designed by the probe design manager of systems of the invention. The probe design manager can design a probe set de novo (e.g., for an identified target), by choosing probes (or probe sets) from a probe database, by accepting all or part of a probe set from a user, or any combination thereof. In general, the probe design manager readies a probe set for filtering by the probe filter manager to produce a “probe group”. By “probe group” is meant one or more probes returned from the probe filter manager by applying one or more probe filters to a probe set.

The term “ligand” as used herein refers to a moiety that is capable of covalently or otherwise chemically binding a compound of interest. The arrays of solid-supported ligands produced by the methods can be used in screening or separation processes, or the like, to bind a component of interest in a sample. The term “ligand” in the context of the invention may or may not be an “oligomer” as defined above. However, the term “ligand” as used herein may also refer to a compound that is “pre-synthesized” or obtained commercially, and then attached to the substrate.

The term “sample” as used herein relates to a material or mixture of materials, typically, although not necessarily, in fluid form, containing one or more components of interest.

A biomonomer fluid or biopolymer fluid refers to a liquid containing either a biomonomer or biopolymer, respectively (typically in solution).

The term “peptide” as used herein refers to any polymer compound produced by amide formation between an α-carboxyl group of one amino acid and an α-amino group of another group.

The term “oligopeptide” as used herein refers to peptides with fewer than about 10 to 20 residues, i.e., amino acid monomeric units.

The term “polypeptide” as used herein refers to peptides with more than 10 to 20 residues.

The term “protein” as used herein refers to polypeptides of specific sequence of more than about 50 residues.

The term “nucleic acid” as used herein means a polymer composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” as used herein denotes single-stranded nucleotide multimers of from about 10 up to about 200 nucleotides in length, e.g., from about 25 to about 200 nt, including from about 50 to about 175 nt, e.g. 150 nt in length

The term “polynucleotide” as used herein refers to single- or double-stranded polymers composed of nucleotide monomers of generally greater than about 100 nucleotides in length.

An “array,” or “chemical array” used interchangeably includes any one-dimensional, two-dimensional or substantially two-dimensional (as well as a three-dimensional) arrangement of addressable regions bearing a particular chemical moiety or moieties (such as ligands, e.g., biopolymers such as polynucleotide or oligonucleotide sequences (nucleic acids), polypeptides (e.g., proteins), carbohydrates, lipids, etc.) associated with that region. As such, an addressable array includes any one or two or even three-dimensional arrangement of discrete regions (or “features”) bearing particular biopolymer moieties (for example, different polynucleotide sequences) associated with that region and positioned at particular predetermined locations on the substrate (each such location being an “address”). These regions may or may not be separated by intervening spaces. In the broadest sense, the arrays of many embodiments are arrays of polymeric binding agents, where the polymeric binding agents may be any of: polypeptides, proteins, nucleic acids, polysaccharides, synthetic mimetics of such biopolymeric binding agents, etc. In many embodiments of interest, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but are generally attached at one of their termini (e.g. the 3′ or 5′ terminus). Sometimes, the arrays are arrays of polypeptides, e.g., proteins or fragments thereof.

Any given substrate may carry one, two, four or more or more arrays disposed on a front surface of the substrate. Depending upon the use, any or all of the arrays may be the same or different from one another and each may contain multiple spots or features. A typical array may contain more than ten, more than one hundred, more than one thousand more ten thousand features, or even more than one hundred thousand features, in an area of less than 20 cm² or even less than 10 cm². For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, or 20% of the total number of features). Interfeature areas will typically (but not essentially) be present which do not carry any polynucleotide (or other biopolymer or chemical moiety of a type of which the features are composed). Such interfeature areas typically will be present where the arrays are formed by processes involving drop deposition of reagents but may not be present when, for example, light directed synthesis fabrication processes are used. It will be appreciated though, that the interfeature areas, when present, could be of various sizes and configurations.

Each array may cover an area of less than 100 cm², or even less than 50 cm², 10 cm² or 1 cm². In many embodiments, the substrate carrying the one or more arrays will be shaped generally as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, the substrate may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays may be fabricated using drop deposition from pulse jets of either precursor units (such as nucleotide or amino acid monomers) in the case of in situ fabrication, or the previously obtained biomolecule, e.g., polynucleotide. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. Other drop deposition methods can be used for fabrication, as previously described herein.

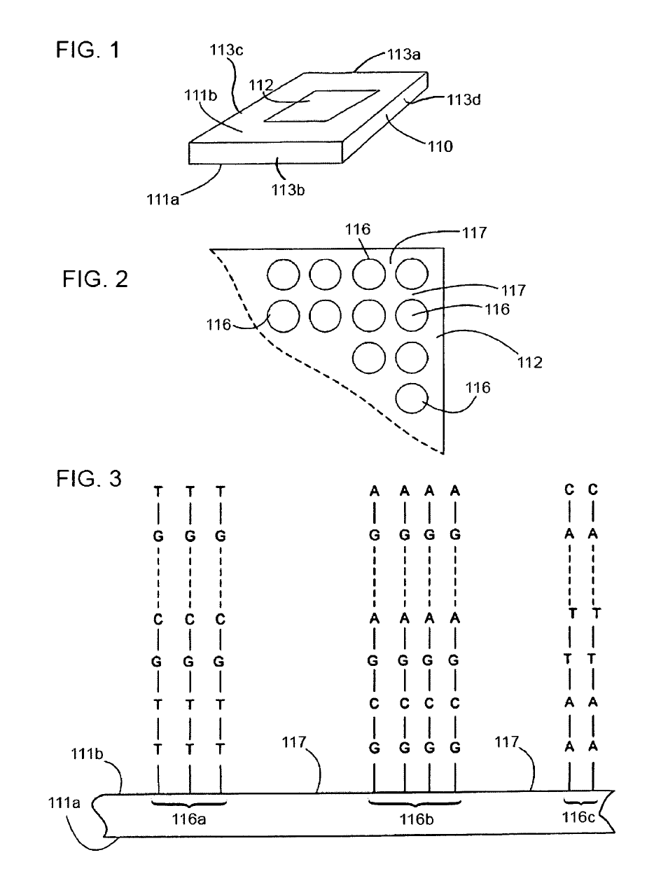

Elements of an exemplary chemical array is shown in FIGS. 1, 2 and 3, where the array shown in this representative embodiment includes a contiguous planar substrate 110 carrying an array 112 disposed on a surface 111b of substrate 110. It will be appreciated though, that more than one array (any of which are the same or different) may be present on surface 111b, with or without spacing between such arrays. That is, any given substrate may carry one, two, four or more arrays disposed on a front surface of the substrate and depending on the use of the array, any or all of the arrays may be the same or different from one another and each may contain multiple spots or features. The one or more arrays 112 usually cover only a portion of the surface 111b, with regions of the rear surface 111b adjacent the opposed sides 113c, 113d and leading end 113a and trailing end 113b of slide 110, not being covered by any array 112. A second surface 111a of the slide 110 does not carry any arrays 112. Each array 112 can be designed for testing against any type of sample, whether a trial sample, reference sample, a combination of them, or a known mixture of biopolymers such as polynucleotides. Substrate 110 may be of any shape, as mentioned above.

As mentioned above, array 112 contains multiple spots or features 116 of biopolymer ligands, e.g., in the form of polynucleotides. As mentioned above, all of the features 116 may be different (e.g., 116a, 116b, 116c), or some or all could be the same. The interfeature areas 117 could be of various sizes and configurations. Each feature carries a predetermined biopolymer such as a predetermined polynucleotide (which includes the possibility of mixtures of polynucleotides). It will be understood that there may be a linker molecule (not shown) between the rear surface 111b and the first nucleotide. Any convenient linker may be used.

Substrate 110 may carry on surface 111a, an identification code, e.g., in the form of bar code (not shown) or the like printed on a substrate in the form of a paper label attached by adhesive or any convenient means. The identification code contains information relating to array 112, where such information may include, but is not limited to, an identification of array 112, i.e., layout information relating to the array(s), etc.

The substrate may be porous or non-porous. The substrate may have a planar or non-planar surface.

In those embodiments where an array includes two more features immobilized on the same surface of a solid support, the array may be referred to as addressable. An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “probe” may be the one which is to be evaluated by the other (thus, either one could be an unknown mixture of analytes, e.g., polynucleotides, to be evaluated by binding with the other).

An array “assembly” includes a substrate and at least one chemical array, e.g., on a surface thereof. Array assemblies may include one or more chemical arrays present on a surface of a device that includes a pedestal supporting a plurality of prongs, e.g., one or more chemical arrays present on a surface of one or more prongs of such a device. An assembly may include other features (such as a housing with a chamber from which the substrate sections can be removed). “Array unit” may be used interchangeably with “array assembly”.

The term “substrate” as used herein refers to a surface upon which marker molecules or probes, e.g., an array, may be adhered. Glass slides are the most common substrate for biochips, although fused silica, silicon, plastic and other materials are also suitable.

When two items are “associated” with one another they are provided in such a way that it is apparent one is related to the other such as where one references the other. For example, an array identifier can be associated with an array by being on the array assembly (such as on the substrate or a housing) that carries the array or on or in a package or kit carrying the array assembly. “Stably attached” or “stably associated with” means an item's position remains substantially constant where in certain embodiments it may mean that an item's position remains substantially constant and known.

A “web array substrate” references a long continuous piece of substrate material having a length greater than a width. For example, the web length to width ratio may be at least 5/1, 10/1, 50/1, 100/1, 200/1, or 500/1, or even at least 1000/1.

“Flexible” with reference to a substrate or substrate web, refers to a substrate that can be bent 180 degrees around a roller of less than 1.25 cm in radius. The substrate can be so bent and straightened repeatedly in either direction at least 100 times without failure (for example, cracking) or plastic deformation. This bending must be within the elastic limits of the material. The foregoing test for flexibility is performed at a temperature of 20° C.

“Rigid” refers to a material or structure which is not flexible, and is constructed such that a segment about 2.5 by 7.5 cm retains its shape and cannot be bent along any direction more than 60 degrees (and often not more than 40, 20, 10, or 5 degrees) without breaking.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions.

“Hybridizing” and “binding”, with respect to polynucleotides, are used interchangeably.

The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

“Stringent hybridization conditions” and “stringent hybridization wash conditions” in the context of nucleic acid hybridization (e.g., as in array, Southern or Northern hybridizations) are sequence dependent, and are different under different experimental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1 M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions sets forth the conditions which determine whether a nucleic acid is specifically hybridized to a surface bound nucleic acid. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C.

A specific example of stringent assay conditions is rotating hybridization at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5×SSC and 0.1 ×SSC at room temperature.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions may also be employed, as appropriate.

“Contacting” means to bring or put together. As such, a first item is contacted with a second item when the two items are brought or put together, e.g., by touching them to each other.

“Depositing” means to position or place an item at a location, or otherwise cause an item to be so positioned or placed at a location. Depositing includes contacting one item with another. Depositing may be manual or automatic, e.g., “depositing” an item at a location may be accomplished by automated robotic devices.

By “remote location,” it is meant a location other than the location at which the array (or referenced item) is present and hybridization occurs (in the case of hybridization reactions). For example, a remote location could be another location (e.g., office, lab, etc.) in the same city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart.

“Communicating” information means transmitting the data representing that information as signals (e.g., electrical, optical, radio signals, and the like) over a suitable communication channel (for example, a private or public network).

“Forwarding” an item refers to any means of getting that item from one location to the next, whether by physically transporting that item or otherwise (where that is possible) and includes, at least in the case of data, physically transporting a medium carrying the data or communicating the data.

An array “package” may be the array plus only a substrate on which the array is deposited, although the package may include other features (such as a housing with a chamber).

A “chamber” references an enclosed volume (although a chamber may be accessible through one or more ports). It will also be appreciated that throughout the present application, words such as “top,” “upper,” and “lower” are used in a relative sense only.

It will also be appreciated that throughout the present application, that words such as “cover”, “base” “front”, “back”, “top”, are used in a relative sense only. The word “above” used to describe the substrate and/or flow cell is meant with respect to the horizontal plane of the environment, e.g., the room, in which the substrate and/or flow cell is present, e.g., the ground or floor of such a room.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present, and, thus, the description includes structures wherein a non-hydrogen substituent is present and structures wherein a non-hydrogen substituent is not present.

### DETAILED DESCRIPTION

Systems and methods for filtering a target probe set are provided. The subject systems include a communications module and a processing module, where the processing module includes: (1) a probe design manager configured to design a probe set, wherein said probe set contains probes specific for one or more target nucleic acid sequences; and (2) a probe filter manager configured to: (a) select a probe group from the probe set using a probe filter, where the probe filter comprises a set of rules for selecting the probe group; and (b) communicate the selected probe group to a user. Also provided are computer program products for executing the subject methods.

In certain embodiments, the present invention is implemented on a web-based platform, where by web-based platform is meant that the probe filtering of a probe set is done for a user over the internet. In certain embodiments, the present invention includes an integrated web-based platform that provides a user access to coupled probe design and probe filtering functionalities, both of which can be very complex. This facilitates the creation of one or more probe groups that meet a user's criteria without requiring the user to manipulate a designed probe set and/or filter the probes themselves. As such, aspects of the present invention are more conducive to iterative workflow in designing and filtering a probe set for use in assays for which such probes find use (e.g., comparative genome hybridization, gene expression analysis, FISH, etc.).

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

Aspects of the invention include systems and methods for filtering a probe set for one or more target nucleic acids of interest. Representative embodiments of the subject systems generally include the following components: (a) a communications module for facilitating information transfer between the system and one or more users, e.g., via a user computer, as described below; and (b) a processing module for performing one or more tasks involved in the probe set filtering methods of the invention. In general, the subject systems may be viewed as being the physical embodiment of a web portal, where the term “web portal” refers to a web site or service, e.g., as may be viewed in the form of a web page, that offers a broad array of resources and services to users via an electronic communication element, e.g., via the Internet.

In certain embodiments, the subject systems include components of array development systems, including but not limited to those systems described in Published United States Application publication Nos. 20060116827; 20060116825 and 20060115822, as well as U.S. patent application Ser. Nos. 11/349,425; 11/349,398; 11/478,975; 11/479,014; 11/459,331 and 11/478,973; the disclosures of which are herein incorporated by reference.

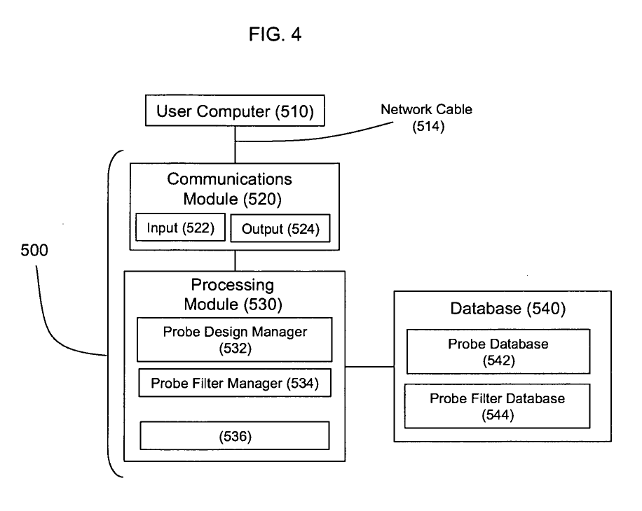

FIG. 4 provides a view of a representative probe set filtering system according to an embodiment of the subject invention. In FIG. 4, system 500 includes communications module 520 and processing module 530, where each module may be present on the same or different platforms, e.g., servers, as described above.

The communications module includes the input manager 522 and output manager 524 functional elements. Input manager 522 receives information from a user e.g., over the Internet. Input manager 522 processes and forwards this information to the processing module 530. These functions are implemented using any convenient method or technique. Another of the functional elements of communications module 520 is output manager 524. Output manager 524 provides information assembled by processing module 530 to a user, e.g., over the Internet. The presentation of data by the output manager may be implemented in accordance with any convenient methods or techniques. As some examples, data may include SQL, HTML or XML documents, email or other files, or data in other forms. The data may include Internet URL addresses so that a user may retrieve additional SQL, HTML, XML, or other documents or data from remote sources.

The communications module 520 may be operatively connected to a user computer 510 via network cable 514, which provides a vehicle for a user to interact with the system 500. User computer 510 may be a computing device specially designed and configured to support and execute any of a multitude of different applications. Computer 510 also may be any of a variety of types of general-purpose computers such as a personal computer, network server, workstation, or other computer platform now or later developed. Computer 510 may include components such as a processor, an operating system, a graphical user interface (GUI) controller, a system memory, memory storage devices, and input-output controllers. There are many possible configurations of the components of computer 510 and some components are not listed above, such as cache memory, a data backup unit, and many other devices.

In certain embodiments, a computer program product is described comprising a computer usable medium having control logic (computer software program, including program code) stored therein. The control logic, when executed by the processor of the computer, causes the processor to perform functions described herein. In other embodiments, some functions are implemented primarily in hardware using, for example, a hardware state machine. Implementation of the hardware state machine so as to perform the functions described herein may be accomplished using any convenient method and techniques.

As described above, a user employs computer 510 to enter information into and retrieve information from the system 500. Additional computers of other users and/or administrators of the system in a local or wide-area network including an Intranet, the Internet, or any other network may also be coupled to system 500 via cable 514. It will be understood that cable 514 is merely representative of any type of network connectivity, which may involve cables, transmitters, relay stations, network servers, wireless communication devices, and many other components not shown suitable for the purpose. Via user computer 510, a user may operate a web browser served by a user-side Internet client to communicate via Internet with system 500. System 500 may similarly be in communication over Internet with other users, networks of users, and/or system administrators, as desired. Embodiments of probe filtering systems of the subject invention that employ internet communication sub systems are referred to herein as being implemented on an internet-based or web-based platform.

As reviewed above, the systems include various functional elements that carry out specific tasks on the platforms in response to information introduced into the system by one or more users. In FIG. 4, elements 532, and 534 and 536 represent three different functional elements of processing module 530. While three different functional elements are shown, it is noted that the number of functional elements may be more or less, depending on the particular embodiment of the invention. Representative functional elements that may be included in the processing module are now reviewed in greater detail below.

In certain embodiments, the subject system includes a probe design manager 532 and probe filter manager 534 as parts of the processing module 530, which is configured to perform functions relating to filtering probe sets of biopolymeric probes for one or more targets (e.g., as specified by a user). Probe design manager 532 is configured to design a set of probes specific the one or more target sequences of interest (discussed in further detail below), where designing includes generating probes de novo (e.g., using a probe design algorithm), selecting probes (or a probe set(s)) from a database, accepting probes from a user, or any combination thereof. In embodiments where a user is specifying a target or targets of interest, the one or more targets can be identified using any convenient method, including but not limited to: uploading target nucleic acid sequence(s), providing one or more target identifier (e.g., gene name, accession numbers, species from which target is derived), selecting from a list presented to the user by the system, etc.

In certain embodiments, the systems include a database 540 configured to store certain data files that may be employed in the probe filtering methods carried out by the systems of the subject invention. In certain embodiment, database 540 includes a probe database 542 comprising a plurality of probe sequences. In certain embodiments, the probe database comprises probe sets that include probe sequences specific for one or more targets. In certain embodiments, the probe sets may be from a previous probe design session (e.g., based on input from a user) or represent probe sets designed by an administrator of the system. The probes in the database may include any number of annotations describing a probe or a set of probes (see definition of annotation above). Probes in the probe database may be derived from other public or private databases, where in certain embodiments the probes have been empirically tested and validated as being specific for a target under certain assay conditions.

In certain embodiments, systems of the subject invention includes a filter database 544 that stores a plurality of probe filters, each probe filter having a set of rules for selecting a probe group from an initial probe set. Each rule in a set of rules for a probe filter can define virtually any limit, range, value, etc., for a characteristic of interest for a probe. Such characteristics include, but are not limited to: probe length parameter, probe distribution parameter (e.g., the distance between the 3′ end of a first probe and the 5′ end of a second, downstream probe), probes per target, probe melting temperature (Tₘ), base composition parameter, sequence quality score, specific nucleotide base % (or X base %), thermodynamic properties, intramolecular hybridization characteristics, intermolecular hybridization characteristics, cross-hybridization potential (either within a species or between species), validation status, biological characteristic, species and hybridization assay parameter.

As noted above, probe design manager 532 is configured to design an initial probe set specific for one or more targets which subsequently will be filtered by the probe filter manager 534. In certain embodiments, the probe design manager may be employed to design a probe set for a target sequence de novo. As such, a probe design manager 532 is provided that is configured to design a probe set when prompted by the user, e.g., in response to a user inputting/identifying one or more target sequence. The probe design manager may employ any convenient probe design algorithm(s) to design a probe(s) for the target sequence. Probe design algorithms of interest include, but are not limited to: those described in U.S. Pat. Nos. 6,251,588 and 6,461,816, as well as published US Application No. 20060110744; the disclosures of which probe design algorithms are incorporated herein by reference. In certain embodiments, the probe design manager operates the design algorithm using default settings for various design parameters. In yet other embodiments, the probe design manager operates the design algorithm using one or more parameters that have been set by a user, e.g., through use of an appropriate graphical user interface (GUI), such that the probe design manager designs probes based in part on one or more parameter provided by said user.

In certain embodiments, the probe design manager is configured to accept one or more previously designed probes from a user to include in a probe set for filtering by the probe filter manager 534 (e.g., in the form of a file uploaded to the system).

In certain embodiments, the probe design manager is configured to choose one or more previously designed probes (or part or all of a probe set) from a probe database to include in a probe set for filtering by the probe filter manager 534.

As such, embodiments of the subject systems include a probe design manager configured to design a probe set by generating probes de novo (e.g., using a probe design algorithm), choosing probes from one or more probe database, accepting probes from a user, or any combination thereof.

A probe set that is to be filtered, sometimes referred to as an initial probe set, can have any number of probes, i.e., 1 or more probes. As such, the number of probes in a probe set for filtering can be 10 or more, 100 or more, 1000 or more, 10,000 or more, 100,000 or more, etc. No upper limit for the number of probes in a probe set to be filtered is intended.

In certain embodiments, the probe design manager is configured to determine one or more characteristic of the probes in a probe set designed by the probe design manager. In certain other embodiments, one or more characteristic of a probe or probe set is determined and provided by a user or an administrator of the system. Characteristics determined by the probe design manager or provided by a user/administrator of the system include, but are not limited to, one or more of the following: probe length parameter, probe melting temperature (Tₘ), base composition parameter, sequence quality score, specific nucleotide base % (or X base %), thermodynamic properties, intramolecular hybridization characteristics, intermolecular hybridization characteristics, cross-hybridization potential (either within a species or between species), species, validation status, biological characteristic of the specific target, where by “biological characteristic” is meant any relevant biological information associated with the specific target for a probe, including cells, tissues, diseases, species, conditions, signal transduction pathways, etc., that are associated with the absence or presence of the expressed target. Any convenient characteristic of a probe a probe set can be provided.

In certain embodiments, a probe set to be filtered is designed for a particular assay to be performed using the probes. Examples of assays include, but are not limited to: comparative genome hybridization (CGH), gene expression analysis (e.g., microarray, or gene chip, assays), single nucleotide polymorphism analysis, chromosomal immuno-precipitation on gene chip (also called “ChIP on Chip assays”), northern blots, southern blots, dot blots, etc.

Once a probe set is designed, probe filter manager 534 selects a probe group from the initial probe set based on a specific probe filter. In certain embodiments, the probe filter to be applied by the probe filter manager is selected by a user of the system. In certain embodiments, a user selects a probe filter by engaging one or more graphical icons presented on a GUI of the system. Any convenient type of graphical icon can be employed, including, but not limited to: drop-down menus, virtual buttons, selectable text strings, fill-in boxes, hyperlinks, single- and multiple-click activated icons or text strings, and any combination thereof. In certain embodiments, a user creates a new probe filter for filtering a probe set by engaging multiple graphical icons on the GUI which together specify the set of rules which make up that probe filter (described in further detail below).

In certain embodiments, the system further includes a user domain for saving information specific for a particular user of the system. In certain embodiments, a user domain of the system is configured to save probe sets designed by the system for the user, probe groups selected by the system based on one or more probe filters, probe filters, and/or array layouts made by the user. In certain embodiments, an activatable graphical icon is displayed to a user that when selected by a user (e.g., by clicking in the icon) indicates that the user wants an item (e.g., probe set, probe group, probe filter, array layout, etc.) to be saved in the user domain. Any convenient method for selecting an item may be employed to have it saved in a user domain. In certain embodiments, the user domain is a memory location contained within the system (500), whereas in other embodiments, the user domain is a memory location in the user computer (510).

In certain embodiments, the user domain is a temporary memory location, e.g., a cache, which is maintained for a specific user during a single session on the system. In these embodiments, once the user exits the system (i.e., once a session is terminated), the information in the user domain is deleted. In certain other embodiments, the user domain is a stable (or long term) memory location that is maintained between user sessions on the system. In certain embodiments, the information stored in a specific user domain can be accessed only by the user who created it. In certain other embodiments, a user domain can be accesses by other users of the system, e.g., other users that have been given access to the user domain either by the user for which the user domain was created or by a system administrator.

In certain embodiments, the system includes a security manager configured to control access to information in the system in a predetermined manner between at least two different users of the system or between a user and an administrator of the system. In certain embodiments, the security manager allows a probe filter to be modified or deleted only by the user or administrator who provided the probe filter to the system. In certain embodiments, a user can designates one or more other users of the system (or an administrator of the system) who can access their probe set, probe group or probe filter information (e.g., stored in their user domain). Access can be confirmed using any convenient access control method, including by requiring a password form a user trying to access information in a second user's user domain, confirming the IP address of the computer a requesting user is employing to access the information in a second user's user domain, etc.

In certain embodiments, the system further includes an array layout developer for developing array layouts based at least in part on a probe group selected using a probe filter according to the present invention. In certain embodiments, the probe group is saved in a user domain. In certain embodiments, the system further includes a fabrication station for fabricating probes and/or arrays having features arranged according to an array layout retrieved from the database of the system and/or developed by the array layout developer.

As noted above, a probe filter includes a set of one or more rules for selecting a probe group from a probe set (or, in certain embodiments, another probe group). Each of the rules in the set of rules in a probe filter defines the limit, range, value, etc., of a characteristic of a probe (or probes) (see, e.g., list of exemplary characteristics above). For example, a rule in a probe filter may define a minimum value, maximum value, average value, value range, or absolute value for a characteristic of one or more probes in a probe set. As such, when a probe filter is applied by the probe filter manager to a probe set designed by the probe design manager, a probe group is returned to the user that meets or exceeds each one of the rules in the set of rules in the corresponding probe filter. It is noted here that in cases where no probes in a probe meet or exceed each one of the rules in an applied probe filter, the system communicates this to the user. In certain embodiments, the probe filter manager is configured to prompt the user to either modify one or more rule in the set of rules in the probe filter that returned no probes from the initial probe set or to select a different probe filter altogether.

In certain embodiments, a user selects a specific probe filter to be a default filter, where by default filter is meant the probe filter that is used to filter a probe set during a probe filter session unless a different filter is selected by the user. In certain embodiments the default filter is a probe filter that was designed by a user, whereas in other embodiments, the default filter is a probe filter designed by an administrator of the system.

In certain embodiments, the probe filter manager returns a hierarchical ranking of some or all of the probes of a probe set using a probe filter. For example, a probe filter may include a rule that ranks probes of a probe set with regard to one or more desired characteristic. For example, a probe filter can include a rule that instructs the probe filter manager to rank the probes in a probe set from lowest to highest Tₘ. Or, a probe filter can include a rule that instructs the probe filter manager to return the 10 probes in a probe set that have the best (or lowest) Tₘ.

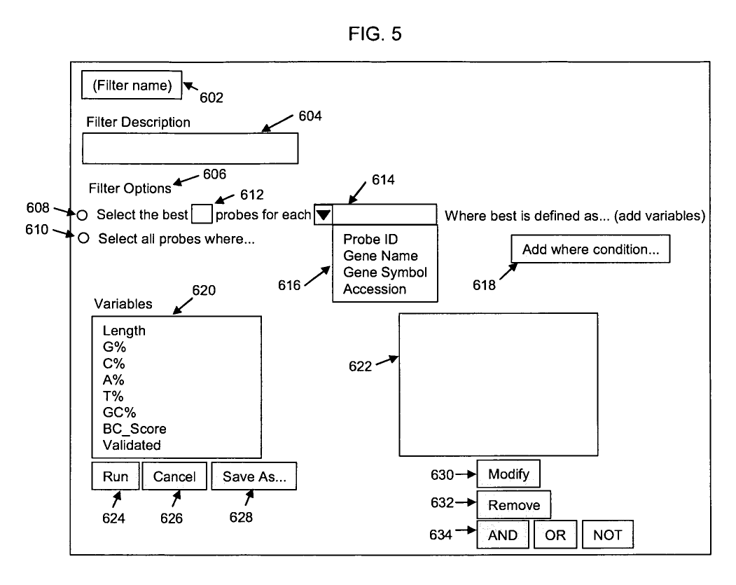

FIG. 5 provides an example of a user interface schematic that is presented to a user on a GUI of the system which is configured to select, design, revise, etc., a probe filter according to certain embodiments of the present invention. It is noted here that the elements shown in FIG. 5 are not meant to be exhaustive.

Element 602 represents a text box that displays the probe filter name currently being viewed. When a new probe filter is being generated by a user of the system, the user can simply fill in a name of their choosing the box (e.g., by selecting the box and typing in the name. In certain embodiments, box 602 may be employed as an input box to search for a probe filter of interest. In certain of these embodiments, a user may enter part or all of a probe filter name and select a search button (not shown) or simply hit the return key. The system will then display probe filters that meet the search term(s); the user can then select the probe filter of interest. In certain embodiments, box 602 is configured to have an auto-fill functionality that presents the name of a probe filter that best fits the probe name being typed into text box 602 in real time (similar to the auto-fill function present in certain web browsers).

Text box 604 provides additional probe filter information (or Filter Description) which describes one or more feature of the probe filter being viewed. This additional information can be virtually any information about the probe filter, including the user/administrator who designed it, parameters it filters, assays for which it is well-suited, etc. As with the Filter Name text box, Filter Description text box 604 box can be filled in by a user when generating a new probe filter, list a Filter Description of a probe filter currently being viewed, or be configured as a search input box.

Items shown below the Filter Options heading 606 relate to displaying components of the specific set of rules for the probe filter being viewed, designed, revised, (etc.), as well as graphical icons for inputting probe filter rule parameters.

Activatable bullets 608 and 610 allow a user to specify the conditions of a specific rule (the “where” conditions). If bullet button 608 is activated, the user inputs the number of probes to be returned into text box 612 for a desired category listed in text box 614 (e.g., by selecting a category from drop-down menu 616) based on a specific probe characteristic (e.g., by defining the “where” condition). The user specifies the “where” condition by highlighting a Variable in box 620 and activating button 618. This action leads to the display on the GUI of a query text box requesting the user to input a value (e.g., minimum, maximum, range, average, etc.) for the particular Variable selected (not shown in FIG. 5). Once the user provides this value, a new probe filter rule is entered into rule box 622 by the system, where the probe filter rule placed in box 622 includes all relevant information selected/input by the user. For example, if the number 5 is place in box 612, “Gene Name” is placed in box 614, G % is selected in the Variables box 620, and the value input by the user is “between 25 and 30%”, then a new probe filter rule is added to box 622 that reads “return 5 probes for each Gene name that each have a G % of between 25 and 30%”.

If bullet 610 is activated, the user again specifies rules by activating button 618, selecting a variable in box 620, and providing a value for the variable. A new probe filter rule is added to box 622 which includes the relevant information.

The buttons listed under box 622 (i.e., Modify (630), Remove (632) and the Boolean linkers AND, OR and NOT (634)) allow a user to build and edit a probe filter rule set having any number of probe filter rules in any desired relational configuration. Modify button 630 allows a user to select a specific probe filter rule in box 622 and modify one or more of its parameters. Remove button 632 allows a user to remove a probe filter rule from probe filter rule box 622. Boolean buttons 634 allow a user to specify how a probe filter rule should be implemented with regard to other rules in probe filter rule box 622. For example, using the graphical icons on FIG. 5, a user could generate a probe filter set that returns all probes that: 1) have a GC % between 20 and 40% AND a length between 15 and 35 nucleotides; OR 2) that are validated. Once a probe filter has been selected, designed, and/or revised to the satisfaction of a user, it can be applied to a probe set by activating Run button 624. In certain embodiments, activation of Run button 624 will generate a selection window or other selectable graphical icon that requests the user to select a probe set to which the displayed probe filter is to be applied (e.g., probe sets saved in a user domain in the system). In certain other embodiments, the probe set of interest has already been selected, and activating Run button 624 will immediately apply the displayed probe filter to the designed probe set of interest.

A probe filter can be saved by the system, e.g., in the probe filter database and/or a user domain, by selecting the “Save As . . . ” button 628. Activating this button may present a window to the user that will allow selection of the location in which to store the probe filter. In certain embodiments, the Filter name can be changed at this step.

Selection of “Cancel” button 626 results in deletion of the information displayed in the current probe filter on the GUI. In certain embodiments, the probe filter manager is configured to present a window to the user to confirm that all information is to be removed from the currently displayed probe filter. In certain embodiments, the probe filter manager asks the user whether to remove the displayed probe filter permanently from the system, e.g., from the user domain and/or the probe filter database.

In certain embodiments, a probe filter system according to the present invention is implemented with an existing probe selection, probe design, and/or array layout design system. In such embodiments, a probe filter can be selected and applied at any number of points along the probe design/selection process. In certain embodiments, a GUI used in implementing a probe filter system of the present invention displays a graphical icon to a user (a Probe Filter Icon), which, when selected, will apply a specified probe filter to the probe set currently designed/selected. In certain embodiments, the user has already specified which probe filter to apply whereas in other embodiments, a selection window is presented to the user upon activation of the probe filter icon that allows the user to specify the probe filter (e.g., either by selecting from a menu, searching, or typing in a Filter name). In certain embodiments, when a user activates the Probe Filter icon, the system displays a probe filter schematic (e.g. as shown in FIG. 5), which the user employs to design, select, modify or search for the filter to use.

Probe sets obtained according to the subject systems and methods find use in a variety of different applications, where such applications include, but are not limited to, analyte detection applications in which the presence of a particular analyte in a given sample is detected at least qualitatively, if not quantitatively. Analyte detection methods include, but are not limited to, northern blots, western blots, dot blots, southern blots, etc.

In certain embodiments, probe sets using the subject system and methods are employed in a chemical array format. Any convenient method for carrying out assays employing a chemical array(s) may be used. In certain of such methods, the sample suspected of comprising the analyte of interest is contacted with an array of immobilized probes obtained according to the subject methods under conditions sufficient for the analyte to bind to the probe. Thus, if the analyte of interest is present in the sample, it binds to the array at the site of its cognate probe and a complex is formed on the array surface. The presence of this binding complex on the array surface is then detected, e.g. through use of a signal production system, e.g. an isotopic or fluorescent label present on the analyte, etc. The presence of the analyte in the sample is then deduced from the detection of binding complexes on the substrate surface.

Specific analyte detection applications of interest include hybridization assays in which the nucleic acid arrays of the subject invention are employed. In these assays, a sample of target nucleic acids is first prepared, where preparation may include labeling of the target nucleic acids with a label, e.g. a member of a signal producing system. Following sample preparation, the sample is contacted with the array under hybridization conditions, whereby complexes are formed between target nucleic acids that are complementary to probe sequences attached to the array surface. The presence of hybridized complexes is then detected. Specific hybridization assays of interest which may be practiced using the subject arrays include: gene discovery assays, differential gene expression analysis assays; nucleic acid sequencing assays, and the like. Patents and patent applications describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992. Also of interest are U.S. Pat. Nos. 6,656,740; 6,613,893; 6,599,693; 6,589,739; 6,587,579; 6,420,180; 6,387,636; 6,309,875; 6,232,072; 6,221,653; and 6,180,351. In certain embodiments, the subject methods include a step of transmitting data from at least one of the detecting and deriving steps, as described above, to a remote location.

Where the arrays are arrays of polypeptide binding agents, e.g., protein arrays, specific applications of interest include analyte detection/proteomics applications, including those described in U.S. Pat. Nos. 4,591,570; 5,171,695; 5,436,170; 5,486,452; 5,532,128 and 6,197,599 as well as published PCT application Nos. WO 99/39210; WO 00/04832; WO 00/04389; WO 00/04390; WO 00/54046; WO 00/63701; WO 01/14425 and WO 01/40803—the disclosures of which are herein incorporated by reference.

As such, in using an array having probes obtained by the system and method of the present invention, the array will typically be exposed to a sample (for example, a fluorescently labeled analyte, e.g., protein containing sample) and the array then read. Reading of the array may be accomplished by illuminating the array and reading the location and intensity of resulting fluorescence at each feature of the array to detect any binding complexes on the surface of the array. For example, a scanner may be used for this purpose which is similar to the AGILENT MICROARRAY SCANNER available from Agilent Technologies, Palo Alto, Calif. Other suitable apparatus and methods are described in U.S. Pat. Nos. 5,091,652; 5,260,578; 5,296,700; 5,324,633; 5,585,639; 5,760,951; 5,763,870; 6,084,991; 6,222,664; 6,284,465; 6,371,370 6,320,196 and 6,355,934. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample or an organism from which a sample was obtained exhibits a particular condition). The results of the reading (processed or not) may be forwarded (such as by communication) to a remote location if desired, and received there for further use (such as further processing).

In certain embodiments, the systems may include additional functionalities. For example, in certain embodiments the systems are employed in the generation of array layouts, where the probes qualified and/or redesigned by the systems are employed. In such embodiments, the array layouts generated by the subject systems can be layouts for any type of chemical array, where in certain embodiments the array layouts are layouts for biopolymeric arrays, such as nucleic acid and amino acid arrays. In certain embodiments, the layouts generated by the subject systems are for nucleic acid arrays.

In certain embodiments, the systems include an array layout functionality, e.g., as described in copending application Ser. No. 11/001,700. In certain of these embodiments, the system includes an array layout developer, where the array layout developer includes a memory having a plurality of rules relating to array layout design and is configured to develop an array layout based on the application of one or more of the rules to information that includes array request information received from a user.

In certain embodiments, the output manager further provides a user with information regarding how to purchase the identified probe set, e.g., alone or in an array. In certain embodiments, the information is provided in the form of an email. In certain embodiments, the information is provided in the form of web page content on a graphical user interface in communication with the output manager. In certain embodiments, the web page content provides a user with an option to select for purchase one or more synthesized probe sequences. In certain embodiments, the web page content includes fields for inputting customer information. In certain embodiments, the system can store the customer information in the memory. In certain embodiments, the customer information includes one or more purchase order numbers. In certain embodiments, the customer information includes one or more purchase order numbers and the system prompts a user to select a purchase order number prior to purchasing the one or more synthesized probe sequences.

In certain embodiments, in response to the purchasing, the one or more probe sequences of probe set are synthesized on an array. In certain embodiments, the methods include ordering synthesized probe(s) that include the sequences of the selected probe group. In certain embodiments, the synthesized probes are synthesized on an array. In certain embodiments, the inputting is via a graphical user interface in communication with the system.

In certain embodiments, the user may choose to obtain an array having the generated probe(s) present therein. As such, the generated probe can be included in an array layout, and an array fabricated according to the array layout that includes the generated probe. In certain embodiments, the user may specify the location of the probe in the product layout. Specifying may include choosing a particular location in a given layout, or choosing from a section of system-provided array layout options in which the probe is present at various locations. Array fabrication according to an array layout can be accomplished in a number of different ways. With respect to nucleic acid arrays in which the immobilized nucleic acids are covalently attached to the substrate surface, such arrays may be synthesized via in situ synthesis in which the nucleic acid ligand is grown on the surface of the substrate in a step-wise fashion and via deposition of the full ligand, e.g., in which a presynthesized nucleic acid/polypeptide, cDNA fragment, etc., onto the surface of the array.

Where the in situ synthesis approach is employed, conventional phosphoramidite synthesis protocols are typically used. In phosphoramidite synthesis protocols, the 3′-hydroxyl group of an initial 5′-protected nucleoside is first covalently attached to the polymer support, e.g., a planar substrate surface. Synthesis of the nucleic acid then proceeds by deprotection of the 5′-hydroxyl group of the attached nucleoside, followed by coupling of an incoming nucleoside-3′-phosphoramidite to the deprotected 5′ hydroxyl group (5′-OH). The resulting phosphite triester is finally oxidized to a phosphotriester to complete the internucleotide bond. The steps of deprotection, coupling and oxidation are repeated until a nucleic acid of the desired length and sequence is obtained. Optionally, a capping reaction may be used after the coupling and/or after the oxidation to inactivate the growing DNA chains that failed in the previous coupling step, thereby avoiding the synthesis of inaccurate sequences.

In the synthesis of nucleic acids on the surface of a substrate, reactive deoxynucleoside phosphoramidites are successively applied, in molecular amounts exceeding the molecular amounts of target hydroxyl groups of the substrate or growing oligonucleotide polymers, to specific cells of the high-density array, where they chemically bond to the target hydroxyl groups. Then, unreacted deoxynucleoside phosphoramidites from multiple cells of the high-density array are washed away, oxidation of the phosphite bonds joining the newly added deoxynucleosides to the growing oligonucleotide polymers to form phosphate bonds is carried out, and unreacted hydroxyl groups of the substrate or growing oligonucleotide polymers are chemically capped to prevent them from reacting with subsequently applied deoxynucleoside phosphoramidites. Optionally, the capping reaction may be done prior to oxidation.

With respect to actual array fabrication, in certain embodiments, the user may himself produce an array having the generated array layout. In yet other embodiments, the user may forward the array layout to a specialized array fabricator or vendor, which vendor will then fabricate the array according to the array layout.

In yet other embodiments, the system may be in communication with an array fabrication station, e.g., where the system operator is also an array vendor, such that the user may order an array directly through the system. In response to receiving an order from the user, the system will forward the array layout to a fabrication station, and the fabrication station will fabricate the array according to the forwarded array layout.

Arrays can be fabricated using drop deposition from pulsejets of either polynucleotide precursor units (such as monomers) in the case of in situ fabrication, or the previously obtained polynucleotide. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. Other drop deposition methods can be used for fabrication, as previously described herein. Also, instead of drop deposition methods, light directed fabrication methods may be used, as are known in the art. Interfeature areas need not be present particularly when the arrays are made by light directed synthesis protocols.

The invention also provides programming, e.g., in the form of computer program products, for use in practicing the probe annotation methods of the invention. Programming according to the present invention can be recorded on computer readable media, e.g., any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. Any convenient medium or storage method can be used to create a manufacture that includes a recording of the present programming/algorithms for carrying out the above described methodology.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A system for selecting a probe group, said system comprising:
(A) a communication module comprising an input manager for receiving input from a user and an output manager for communicating output to a user;
(B) a processing module comprising:
(1) a probe design manager configured to design a probe set, wherein said probe set comprises probes specific for one or more target nucleic acid sequences; and
(2) a probe selection manager configured to:
(a) select a probe group from said probe set using a probe filter, wherein said probe filter comprises a set of rules for selecting said probe group; and
(b) communicate said selected probe set to said user;
wherein said system is implemented on a web-based platform.

2. The system of claim 1, wherein said set of rules defines one or more of the following: probe length parameter, probe distribution parameter, probes per target, probe melting temperature (Tm), base composition parameter, sequence quality score, X base %, thermodynamic properties, intramolecular hybridization characteristics, intermolecular hybridization characteristics, cross-hybridization potential, validation status, biological characteristic, and hybridization assay parameter.

3. The system of claim 1, wherein said system further comprises a probe filter database comprising a plurality of probe filters, wherein said probe filter is selected from said probe filter database.

4. The system of claim 3, wherein said plurality of probe filters in said probe filter database are provided to said system by any combination of: said user, an administrator of said system, and any other user of said system.

5. The system of claim 4, wherein said system further comprises a security manager configured to allow a probe filter to be modified or deleted only by the user or administrator who provided said probe filter to said system.

6. The system of claim 1, wherein said system further comprises a graphical user interface (GUI), wherein said probe filter is selected by said user by engaging one or more graphical icons present on said GUI.

7. The system of claim 6, wherein said one or more graphical icons include: drop-down menus, buttons, text strings, fill-in boxes, arrow buttons, and hyperlinks.

8. The system of claim 1, wherein said probe filter employed is selected by said user.

9. The system of claim 8, wherein said probe filter employed is a default filter.

10. The system of claim 1, wherein said system is further configured to select a second probe group from said selected probe group using a second probe filter when prompted by said user.

11. The system of claim 1, wherein said system further comprises a user domain manager configured to save said selected probe group in a user domain specific for said user when prompted by said user.

12. The system of claim 11, wherein said probe set is saved in said user domain when prompted by said user.

13. The system of claim 1, wherein said processing module further comprises an array layout developer configured to develop an array layout comprising said selected probe group.

14. A method of receiving a selected probe set, said method comprising:
(a) providing one or more target nucleic acids to the system of claim 1; and
(b) receiving a selected probe group for said one or more targets.

15. A method of providing a probe group to a user, said method comprising:
(a) designing a probe set based on input received from a user, wherein said probe set comprises probes specific for one or more target nucleic acid sequences;
(b) selecting a probe group from said probe set using a probe filter, wherein said probe filter is:
(i) selected by said user; and
(ii) comprises a set of rules for selecting said probe group;
(c) providing said probe group to said user;
wherein said method is carried out on a web-based platform.

16. The method of claim 15, wherein said set of rules defines one or more of the following: probe length parameter, probe distribution parameter, probes per target, probe melting temperature (Tm), base composition parameter, sequence quality score, X base %, thermodynamic properties, intramolecular hybridization characteristics, intermolecular hybridization characteristics, cross-hybridization potential, validation status, biological characteristic, and hybridization assay parameter.

17. The method of claim 15, wherein said probe filter is selected from a probe filter database comprising a plurality of probe filters.

18. The method of claim 17, wherein each of said plurality of probe filters in said probe filter database is provided by any combination of: said user, an administrator of said system, and any other user of said system.

19. The method of claim 15, wherein said web-based platform comprises a graphical user interface (GUI) that displays graphical icons to said user, wherein said user engages one or more of said graphical icons to select said probe filter.

20. The method of claim 19, wherein said one or more graphical icons include: drop-down menus, buttons, text strings, fill-in boxes, arrow buttons, and hyperlinks.

21. The method of claim 15, wherein said method further comprises selecting a second probe group from said selected probe group using a second probe filter when prompted by said user.

22. The method of claim 15, wherein said selected probe group is saved in a user domain specific for said user when prompted by said user.

23. The method of claim 22, wherein said probe set is saved in said user domain when prompted by said user.

24. The method of claim 15, wherein said method further comprises developing an array layout comprising said selected probe group when prompted by said user.

25. The method of claim 24, wherein said method further comprises fabricating an array based on said array layout and sending said fabricated array to said user.

26. A computer program product comprising a computer readable storage medium having a computer program stored thereon, wherein said computer program, when loaded onto a computer, operates said computer to:
(a) designing a probe set based on input received from a user, wherein said probe set comprises probes specific for one or more target nucleic acid sequences;
(b) selecting a probe group from said probe set using a probe filter, wherein said probe filter is:
(i) selected by said user; and
(ii) comprises a set of rules for selecting said probe group;
(c) providing said probe group to said user;
wherein said steps are carried out on a web-based platform.
